(19) United States
(12) Patent Application Publication
(10) Pub. No.: US 2021/0310984 A1
(43) Pub. Date: Oct. 7, 2021

(54) AMMONIA SENSOR

(71) Applicant: DENSO CORPORATION, Kariya-city (JP)

(72) Inventors: Satoshi NAKAMURA, Nisshin-city (JP); Toshihiko HARADA, Nisshin-city (JP); Kensuke TAKIZAWA, Kariya-city (JP); Hiroki ICHIKAWA, Kariya-city (JP)

(21) Appl. No.: 17/350,143

(22) Filed: Jun. 17, 2021

Related U.S. Application Data
(63) Continuation of application No. PCT/JP2019/047984, filed on Dec. 9, 2019.

(30) Foreign Application Priority Data
Dec. 21, 2018 (JP) 2018-239783

(51) Int. Cl. G01N 27/407; G01N 27/406; G01N 27/41

(52) U.S. Cl. CPC G01N 27/4075; G01N 27/4071; G01N 27/41; G01N 27/4067

(57) ABSTRACT

An ammonia sensor has an ammonia element section having a first solid electrolyte body which is provided with a detection electrode and a reference electrode, a heater section that heats the first solid electrolyte body, and a potential difference detection section for detecting a potential difference between the detection electrode and the reference electrode. The detection electrode contains at least Au and Pd. The content ratio of Au and Pd on the surface of the detection electrode is more than 0 mol % and 80 mol % or less of Pd with respect to 100 mol % of Au.

### CROSS-REFERENCE TO RELATED APPLICATION

The present application is a continuation application of International Application No. PCT/JP2019/047984 filed on Dec. 9, 2019, which is based on and claims the benefit of priority from Japanese Patent Application No. 2018-239783 filed on Dec. 21, 2018. The contents of these applications are incorporated herein by reference in their entirety.

### BACKGROUND

The present disclosure relates to an ammonia sensor equipped with an ammonia element section.

For example, a catalyst for purifying the exhaust gas from a diesel engine which is an internal combustion engine of a vehicle, by removing NOx (nitrogen oxides) such as NO and NO₂, is disposed in the exhaust pipe of the vehicle. In the case of a selective catalytic reduction catalyst (SCR), ammonia (NH₃) contained in aqueous urea or the like, is attached to the catalyst carrier, in order to reduce the NOx by chemically reacting ammonia and NOx on the catalyst carrier. In the reaction, the NOx is reduced to nitrogen (N₂) and water (H₂O).

In addition, a reducing agent supply device for supplying ammonia as a reducing agent to the selective reduction catalyst is disposed in the exhaust pipe, at a position upstream of the flow of the exhaust gas from the selective reduction catalyst. Furthermore, for example, a NOx sensor for detecting the NOx concentration in the exhaust gas and an ammonia sensor for detecting the ammonia concentration in the exhaust gas are disposed in the exhaust pipe, at a position downstream of the flow of the exhaust gas from the selective reduction catalyst.

In the ammonia sensor which detects the ammonia concentration in exhaust gas as the detection target gas, a detection electrode that is exposed to the exhaust gas, and a reference electrode that serves as a reference for obtaining the potential of the detection electrode, are respectively disposed on a solid electrolyte body. The ammonia concentration in the exhaust gas is obtained using a potential difference that is generated between the detection electrode and the reference electrode.

### SUMMARY

One aspect of the present disclosure is an ammonia sensor comprising

an ammonia element section having an oxygen ion conductive solid electrolyte body, a detection electrode and a reference electrode provided on a surface of the solid electrolyte body,

wherein the detection electrode contains at least Au and Pd, and the content proportion of Pd on the surface of the detection electrode, with respect to 100 mol % of Au, is 80 mol %.

It should be noted that the reference signs in parentheses for respective components in an embodiment of the present disclosure indicate the correspondence between the components and reference signs shown in the drawings of the embodiment, however the components are not limited to the contents of the embodiment.

### BRIEF DESCRIPTION OF THE DRAWINGS

The objectives, features and advantages of the present disclosure are made clearer by the following detailed description, referring to the appended drawings. In the drawings:

FIG. 1 is a cross-sectional view showing an ammonia sensor of the first embodiment.

FIG. 2 is a cross-sectional view of the sensor element of the first embodiment taken along the line II-II shown in FIG. 1.

FIG. 3 is a cross-sectional view of the sensor element of the first embodiment taken along the line III-III shown in FIG. 1.

FIG. 4 is a cross-sectional view of the sensor element of the first embodiment taken along the line IV-IV shown in FIG. 1.

FIG. 5 is an explanatory diagram of the electrical configuration of a sensor control unit of the first embodiment.

FIG. 6 is an explanatory diagram showing the arrangement of a gas sensor of the first embodiment in an internal combustion engine.

FIG. 7 is an explanatory diagram showing a hybrid potential generated at the detection electrode according to the first embodiment.

FIG. 8 is an explanatory diagram according to the first embodiment showing a hybrid potential generated in a detection electrode when the ammonia concentration changes.

FIG. 9 is an explanatory diagram according to the first embodiment showing a hybrid potential generated in the detection electrode when the oxygen concentration changes.

FIG. 10 is an explanatory diagram according to the first embodiment showing a hybrid potential generated at the detection electrode when the temperature of the detection electrode changes.

FIG. 11 is a graph according to the first embodiment showing the relationship between the temperature of the detection electrode and an amount of correction of a potential difference.

FIG. 12 is an explanatory diagram according to the first embodiment showing a hybrid potential generated at the detection electrode when the measurement gas contains other gases such as CO and C₃H₈.

FIG. 13 is a graph according to the first embodiment showing a relationship between the ammonia concentration and a potential difference when the oxygen concentration changes.

FIG. 14 is a graph according to the first embodiment showing a relationship between the potential difference and the oxygen concentration after oxygen correction, when the oxygen concentration changes.

FIG. 15 is a cross-sectional view showing a plurality of measurement sites on a cut surface of the detection electrode according to the first embodiment.

FIG. 16 is a graph according to the first embodiment showing a change in sensor output when the ammonia concentration changes.

FIG. 17 is a graph s according to the first embodiment showing the amounts of variation with time of the sensor output when the content proportion of Pd with respect to 100 mol % of Au on the surface of the detection electrode is changed.

FIG. 18 is a graph according to the first embodiment showing sensitivities of the sensor output when the content proportion of Pd with respect to Au: 100 mol % on the surface of the detection electrode is changed.

FIG. 19 is a graph showing values of a variation-with-time index with respect to changes in the content proportion of Pd with respect to 100 mol % of Au on the surface of the detection electrode according to the first embodiment.

FIG. 20 is a cross-sectional explanatory view showing the configuration of an ammonia sensor according to a second embodiment.

FIG. 21 is a cross-sectional explanatory view showing a sensor element according to a third embodiment.

FIG. 22 is a cross-sectional view taken along the line XXII-XXII of FIG. 21, showing the configuration of an ammonia sensor according to the third embodiment.

### DETAILED DESCRIPTION OF THE PREFERRED EMBODIMENTS

A reducing agent supply device for supplying ammonia as a reducing agent to the selective reduction catalyst is disposed in the exhaust pipe, at a position upstream of the flow of the exhaust gas from the selective reduction catalyst. Furthermore, for example, a NOx sensor for detecting the NOx concentration in the exhaust gas and an ammonia sensor for detecting the ammonia concentration in the exhaust gas are disposed in the exhaust pipe, at a position downstream of the flow of the exhaust gas from the selective reduction catalyst. By using the NOx sensor and the ammonia sensors to monitor the amounts of NOx and ammonia, the rate of purification of NOx by ammonia can be improved while suppressing the outflow of ammonia from the selective reduction catalyst.

In the ammonia sensor which detects the ammonia concentration in exhaust gas as the detection target gas, a detection electrode that is exposed to the exhaust gas, and a reference electrode that serves as a reference for obtaining the potential of the detection electrode, are respectively disposed on a solid electrolyte body. The ammonia concentration in the exhaust gas is obtained using a potential difference that is generated between the detection electrode and the reference electrode.

For example, as disclosed in JP 2017-194439 A, in configuring a hybrid potential type of ammonia gas sensor, the detection electrode that is exposed to the gas to be measured is formed with a noble metal consisting of a Pt—Au alloy. According to JP 2017-194439 A, the detection sensitivity and the longevity of the ammonia gas are improved by combining Au with Pt.

Au or Pt—Au is often used as in the detection electrode of an ammonia sensor because of its high sensitivity to ammonia. However, it has been found that if the Au content ratio in the noble metal of the detection electrode is high, then after the ammonia concentration changes to a specific concentration and remains at that specific concentration, the sensor output of the detection electrode, obtained as the potential difference between the detection electrode and the reference electrode, thereafter, changes over time. Research performed by the present inventor has revealed that this variation with time in the sensor output is not caused merely by a deterioration phenomenon of the detection electrode, but by the balance between the adsorption characteristic and the oxidation characteristic of the detection electrode with respect to ammonia.

The adsorption characteristic and oxidation characteristic of the detection electrode with respect to ammonia are indicators of sensitivity in detecting ammonia. If the adsorption characteristic is higher than necessary, then after the sensor output of the detection electrode becomes increased, the variation with time results in further increases in the sensor output. On the other hand, if the oxidation characteristic is higher than necessary, then after the sensor output of the detection electrode becomes decreased, the variation with time results in further decreases in the sensor output. Here, a “variation with time” of the sensor output signifies changes of the sensor output with time that occur after there has been a change in the ammonia concentration. When there is variation with time of the sensor output, a difference arises between the sensor output that is produced immediately after the ammonia concentration has changed to some specific concentration and the sensor output after some time has elapsed since the ammonia concentration changed to that specific concentration, and this is a factor that increases the degree of detection error in the sensor output, that is, it is a factor that lowers the accuracy of detecting the ammonia concentration. It has therefore been found that, to improve the accuracy of detecting the ammonia concentration, it is necessary to appropriately suppress variation with time of the sensor output, by maintaining an appropriate balance between the adsorption characteristic and the oxidation characteristic.

It is an object of the present disclosure to provide an ammonia sensor capable of improved accuracy of detecting an ammonia concentration.

One aspect of the present disclosure is an ammonia sensor comprising

an ammonia element section having an oxygen ion conductive solid electrolyte body, a detection electrode that is provided on a surface of the solid electrolyte body and is exposed to a detection target gas, and having a reference electrode provided on a surface of the solid electrolyte body,

a heater section having a heat generating section that generates heat when energized, and which heats the solid electrolyte body, the detection electrode, and the reference electrode by the generated heat, and

a potential difference detection section that detects a potential difference between the detection electrode and the reference electrode, where the potential difference arises when the electrochemical reduction reaction of oxygen contained in the detection target gas and the electrochemical oxidation reaction of ammonia contained in the detection target gas in the detection electrode are balanced,

wherein the detection electrode contains at least Au and Pd, and the content proportion of Pd on the surface of the detection electrode, with respect to 100 mol % of Au, is 80 mol %.

With the ammonia sensor according to the above aspect, in the configuration for detecting the potential difference between the detection electrode and the reference electrode, the detection electrode contains at least Au (gold) and Pd (palladium), such that the surface of the detection electrode contains Pd in the range of 80 mol % or less with respect to Au of 100 mol %. As a result of research performed by the present inventor, it has been found that if the content ratio of Au and Pd in the noble metal on the surface of the detection electrode is made appropriate, then the adsorption characteristic and oxidation characteristic of the detection electrode with respect to ammonia can be maintained, and variation with time in the sensor output of the detection electrode can be suitably suppressed.

If the content proportion of Pd with respect to Au on the surface of the detection electrode is excessively high, the oxidation characteristic of the detection electrode will increase, the sensitivity of the detection electrode will decrease, and variation with time of the sensor output of the detection electrode will occur. By making the content proportion of Pd greater than 0 mol % and no greater than 80 mol % with respect to 100 mol % of Au, variation with time in the sensor output can be suppressed appropriately while maintaining an appropriate balance between the adsorption characteristic and the oxidation characteristic of the detection electrode.

The adsorption and oxidation characteristics of the detection electrode affect the sensitivity of the detection electrode to ammonia. By increasing the sensitivity of the detection electrode, and making the sensor output of the detection electrode unlikely to vary with time, detection errors are less liable to occur in the sensor output of the detection electrode, and the accuracy of detecting the ammonia concentration by means of the detection electrode can be increased.

Hence, the accuracy of detecting the ammonia concentration can be improved by the ammonia sensor according to the above aspect.

Au on the surface of the detection electrode has a high adsorption characteristic and high sensitivity to ammonia. However, the high adsorption characteristic of the Au causes variation with time to occur in the sensor output, in the positive direction. On the other hand, Pd on the surface of the detection electrode has a high oxidizing characteristic and low sensitivity to ammonia. However, the high oxidation characteristic causes variation with time to occur in the sensor output, in the negative direction. By suitably adjusting the content ratio of Au and Pd on the surface of the detection electrode, an appropriate balance between the adsorption characteristic and the oxidation characteristic can be achieved, enabling variation with time in the sensor output of the detection electrode to be appropriately suppressed.

The adsorption characteristic of the detection electrode with respect to ammonia decreases as the content proportion of Pd with respect to Au on the surface of the detection electrode increases. Lowering of the adsorption characteristic of the detection electrode can be suppressed, and the sensitivity of the detection electrode maintained at a high level, if the content proportion of Pd with respect to Au on the surface of the detection electrode is made as low as possible. The content proportion of Pd with respect to Au can be made 0.5 mol % or more.

On the other hand, the variation with time of the sensor output of the detection electrode tends to occur in the positive direction when the adsorption characteristic of the detection electrode with respect to ammonia is high, and tends to occur in the negative direction when the oxidation characteristic of the detection electrode with respect to ammonia is high. If the sensor output changes significantly over time in the positive direction or negative direction, then a difference is liable to occur between the sensor output immediately after the ammonia concentration has changed to a specific value and the sensor output after a specific time has elapsed since the concentration changed to that specific value.

This variation with time in the sensor output of the detection electrode is smallest when the content proportion of Pd with respect to Au on the surface of the detection electrode is of the order of 20 to 40 mol %. However, it would be possible to further reduce this variation with time by making the content proportion of Pd with respect to Au less than 40 mol %.

The detection electrode surface can have a thickness such as to enable measurement of the content ratio of Au to Pd. For example, the detection electrode surface can be specified as extending to a depth of 1 μm from the outermost face of the detection electrode, as measured in a direction at right angles to the surface of the solid electrolyte body.

The detection electrode may include a noble metal other than Au and Pd, a solid electrolyte body material that is homogenous with the material constituting the solid electrolyte body, or the like. The Au and Pd may be an alloy, or may not be any alloy but mixed with one other. The detection electrode may contain, for example, Pt (platinum) in addition to Au and Pd. The Au, Pd and Pt may be an alloy, or may not be any alloy but mixed with one other.

A preferred embodiment of the above ammonia will be described referring to the drawings.

### First Embodiment

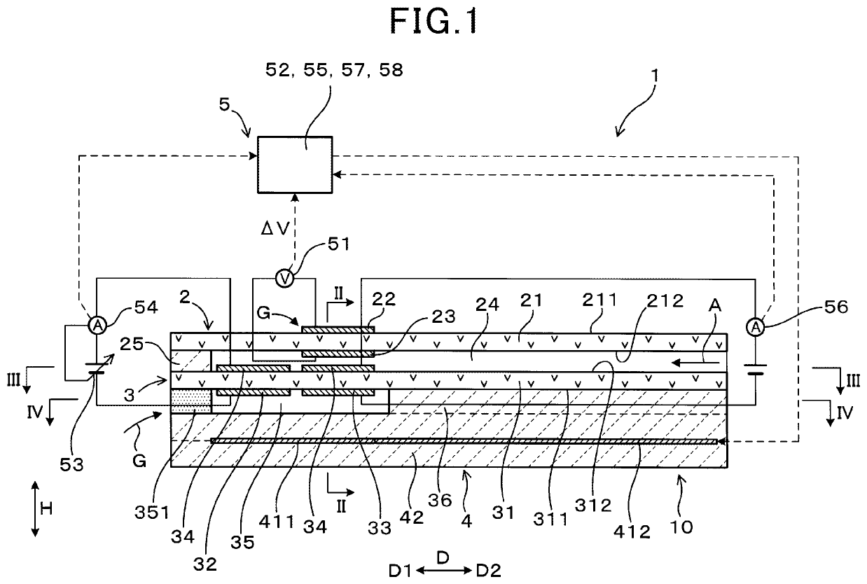

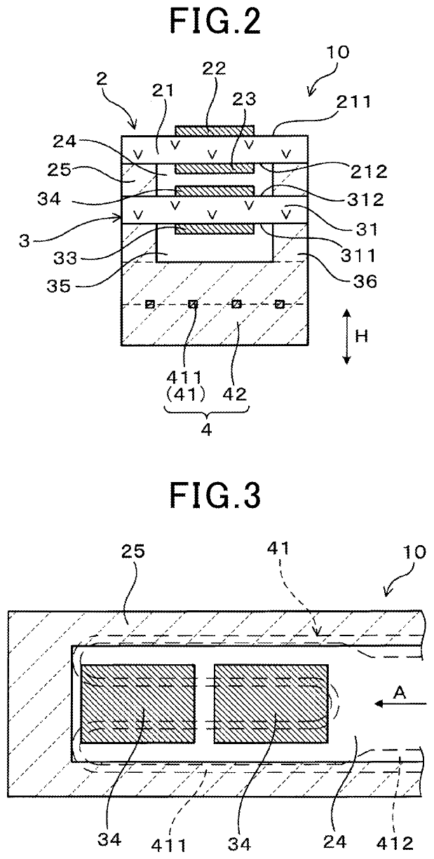

As shown in FIGS. 1 and 2, the ammonia sensor 1 of this embodiment includes an ammonia element section 2, a heater section 4, and a potential difference detection section 51. The ammonia element section 2 and the heater section 4 form part of the sensor element 10. The ammonia element section 2 has a first solid electrolyte body 21 having oxygen ion conductivity, a detection electrode 22 provided on a first surface 211 of the first solid electrolyte body 21 and exposed to a detection target gas G and a reference electrode 23 provided on a second surface 212 of the solid electrolyte body 21. The heater section 4 has a heat generating section 411 that generates heat when energized and can heat the first solid electrolyte body 21, the detection electrode 22, and the reference electrode 23 by the heat generation at the heat generating section 411.

The potential difference detection section 51 detects the potential difference ΔV between the detection electrode 22 and the reference electrode 23 which arises when the electrochemical reduction reaction of oxygen (oxygen gas, O₂) contained in the detection target gas G and the electrochemical oxidation reaction of ammonia (ammonia gas, NH₃) contained in the detection target gas G are balanced. The detection electrode 22 contains at least Au (gold) and Pd (palladium). The content proportion of Au with respect to Pd on the surface of the detection electrode 22 is greater than 0 mol % and less than 80 mol % Pd with respect to 100 mol % of Au.

The ammonia sensor 1 of this embodiment will be described in detail below.

### (Ammonia Sensor 1)

As shown in FIG. 1, the ammonia sensor 1 of this embodiment is of potential difference type, specifically, of hybrid potential type. The concentration of ammonia in a detection target gas G, which is in a state containing oxygen and ammonia, is detected by the ammonia sensor 1. The potential difference detection section 51 of the present embodiment is configured to detect the potential difference ΔV between the detection electrode 22 and the reference electrode 23 which arises when there is a balance between a reduction current due to the electrochemical reduction reaction of oxygen (hereinafter simply referred to as a reduction reaction) and an oxidation current due to the electrochemical oxidation reaction of ammonia (hereinafter simply referred to as an oxidation reaction).

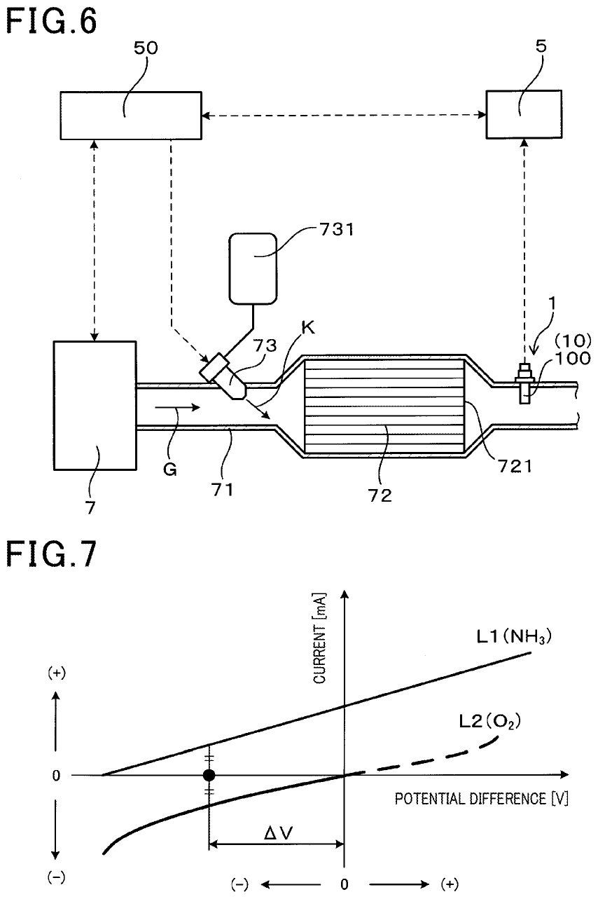

As shown in FIG. 6, the ammonia sensor 1 detects the concentration of ammonia which passes out from a catalyst 72, which reduces NOx, in the exhaust pipe 71 of an internal combustion engine (engine) 7 of a vehicle. The detection target gas G is discharged from the internal combustion engine 7 to the exhaust pipe 71. The composition of the exhaust gas changes depending on the combustion conditions in the internal combustion engine 7. When the air-fuel ratio, which is the mass ratio of air and fuel in the internal combustion engine 7, is in a fuel-rich state compared to a theoretical air-fuel ratio, the composition of the exhaust gas is such the proportion of HC (hydrocarbons), CO (carbon monoxide), H₂ (hydrogen), etc., contained in unburned fuel in the gas is increased, while the proportion of NOx (nitrous oxides) such as NO, NO₂, N₂O is decreased. When the air-fuel ratio in the internal combustion engine 7 is in a fuel lean state as compared with theoretical air-fuel ratio, the proportion of HC, CO, etc. in the composition of the exhaust gas is decreased, while the proportion of NOx is increased. Furthermore, in the fuel-rich state, the detection target gas G contains almost no oxygen (air), while in the fuel lean state, the detection target gas G contains more oxygen (air).

### (Catalyst 72)

As shown in FIG. 6, a catalyst 72 for reducing NOx, and a reducing agent supply device 73 for supplying a reducing agent K containing ammonia to the catalyst 72, are disposed in the exhaust pipe 71. Ammonia is attached to the catalyst carrier of the catalyst 72, as the reducing agent K for NOx. The amount of ammonia adhered to the catalyst carrier of the catalyst 72 becomes decreased by the reduction reaction of NOx. As the amount of ammonia adhering to the catalyst carrier decreases, the reducing agent supply device 73 replenishes the catalyst carrier with ammonia. The reducing agent supply device 73 is disposed in the exhaust pipe 71 at a position upstream of the exhaust gas flow from the catalyst 72, and supplies ammonia gas, generated by injecting aqueous urea, into the exhaust pipe 71. The ammonia gas is produced by hydrolyzing the aqueous urea. An aqueous urea tank 731 is connected to the reducing agent supply device 73.

The internal combustion engine 7 of this embodiment is a diesel engine, which performs a combustion operation by utilizing self-ignition of light oil. The catalyst 72 is a selective reduction catalyst (SCR) that causes NOx (nitrogen oxide) to chemically react with ammonia (NH₃), for reducing the ammonia to nitrogen (N₂) and water (H₂O).

Although not shown, an oxidation catalyst (DOC) that converts NO to NO₂ (oxidation) and reduces CO, HC (hydrocarbon), etc., located upstream of the catalyst 72 in the exhaust pipe 71, and a filter (DPF) for collecting fine particles or the like, may also be provided,

### (Multi-Gas Sensor)

As shown in FIG. 6, the ammonia sensor 1 of this embodiment is disposed downstream from the catalyst 72 in the exhaust pipe 71. Strictly speaking it is the sensor main body 100, which includes the sensor element 10, that is disposed in the exhaust pipe 71, excluding the sensor control unit (SCU) 5 which includes the potential difference detection section 51, etc. The sensor body 100 of this embodiment may be referred to as the ammonia sensor 1, for convenience.

The ammonia sensor 1 of this embodiment is configured as a multi-gas sensor (composite sensor) capable of not only detecting the ammonia concentration but also detecting the oxygen concentration and the NOx concentration. The oxygen concentration is used in the ammonia sensor 1 to correct the ammonia concentration. Furthermore, the ammonia concentration and the NOx concentration that are obtained by the ammonia sensor 1 are used by the engine control unit (ECU) 50, which is the control device of the internal combustion engine 7, to determine the timings at which the reducing agent supply device 73 supplies ammonia as the reducing agent K to the exhaust pipe 71.

The control devices include the engine control unit 50 that controls the engine, a sensor control unit 5 that controls the ammonia sensor 1, and various electronic control units. Here, “control device” refers to various computers (processing devices).

The engine control unit 50 is configured to detect that the catalyst 72 is deficient in ammonia when the ammonia sensor 1 detects that NOx is present in the detection target gas G, and to then inject aqueous urea from the reducing agent supply device 73, to supply ammonia to the catalyst 72. On the other hand, the engine control unit 50 is configured to detect that the catalyst 72 has excessive ammonia when the ammonia sensor 1 detects the presence of ammonia in the detection target gas G, and to then halt the injection of aqueous urea from the reducing agent supply device 73, to thereby halt the supply of ammonia to the catalyst 72. It is preferable that a just enough amount of ammonia is supplied to the catalyst 72 for reducing NOx.

By controlling the supply of ammonia by the engine control unit 50, the states of concentration of NOx and ammonia in the detection target gas G downstream from the catalyst 72 (catalyst outlet 721), at the position of the ammonia sensor 1, enters a state in which the NOx is appropriately reduced by the ammonia, a state in which the amount of NOx outflow increases, or a state in which the amount of ammonia outflow increases, as time passes.

### (Sensor Body 100)

Although not shown in the drawing, the sensor body 100 of the ammonia sensor 1 is provided with a sensor element 10 that detects the ammonia concentration and the NOx concentration and in which a heater section 4 is disposed, a housing attached to the exhaust pipe 71 for retaining the sensor element 10, a tip end side cover attached to the tip end side of the housing to protect the sensor element 10, and a base end side cover attached to the base end side of the housing to protect the electrical wiring part of the sensor element 10. As shown in FIGS. 1 to 3, a heating body 41 constituting the heater section 4 is embedded in the sensor element 10.

### (Sensor Element 10)

To constitute a multi-gas sensor, as shown in FIGS. 1 and 2, the sensor element 10 has an ammonia element section 2 for detecting the ammonia concentration and an oxygen element section 3 for detecting the oxygen concentration and the NOx concentration. The sensor element 10 includes a first solid electrolyte body (solid electrolyte body) 21 constituting the ammonia element section 2, and a second solid electrolyte body (other solid electrolyte body) 31 constituting the oxygen element section 3.

The sensor element 10 of this embodiment is formed in an elongated shape, extending in one direction. A diffusion resistance section 351, described hereinafter, is provided at the tip part of the sensor element 10 with respect to the elongation direction. In FIG. 1, the elongation direction is indicated by an arrow D, the tip end side in the elongation direction D is indicated by an arrow D1, and the base end side in the elongation direction D is indicated by an arrow D2.

The first solid electrolyte body 21 and the second solid electrolyte body 31 are rectangular parallelepipeds formed in a plate shape. Plate-shaped insulators 25, 36, and 42 are stacked on the first solid electrolyte body 21 and the second solid electrolyte body 31. A reference gas duct 24 which houses the reference electrode 23 is formed in the duct insulator 25, positioned between the first solid electrolyte body 21 and the second solid electrolyte body 31. The detection electrode 22 is provided on a first surface 211, which is an outermost surface of the first solid electrolyte body 21, and which forms the outer surface of the sensor element 10 and is exposed to the detection target gas G. The first surface 211 of the first solid electrolyte body 21 forms the outer surface of the sensor element 10 with which detection target gas G is incident at a predetermined flow velocity.

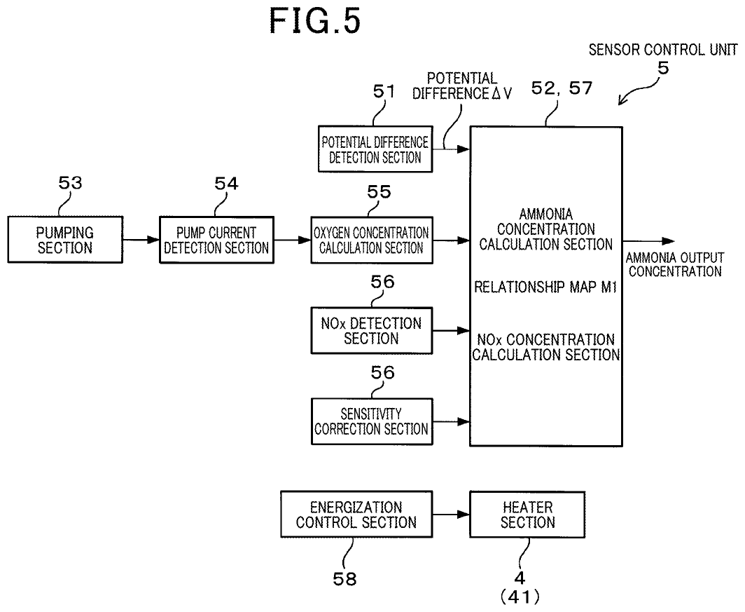

As shown in FIGS. 1 and 5, the ammonia sensor 1 of the present embodiment includes an ammonia concentration calculation section 52 and an energization control section 58 in addition to the ammonia element section 2, the heater section 4, and the potential difference detection section 51. The ammonia concentration calculation section 52 is configured to calculate the ammonia concentration in the detection target gas G based on the oxygen concentration in the detection target gas G and the potential difference ΔV obtained by the potential difference detection section 51, with the ammonia concentration being corrected depending on the oxygen concentration. The energization control section 58 is configured to control the degree of energization of the heating body 41 such that the temperature of the detection electrode 22 becomes a target control value within the range of 400 to 600° C. Furthermore, the ammonia concentration calculation section 52 is configured such that the higher the target control temperature that is set by the energization control section 58, the smaller is made the amount of correction of the ammonia concentration that is applied when the oxygen concentration changes by a specific amount. The heater section 4 has a heating body 41 that generates heat when energized.

### (Ammonia Element Section 2)

As shown in FIGS. 1 and 2, the first solid electrolyte body 21 is formed in a plate shape and is made of a zirconia material having the property of conducting oxygen ions when the material is at a predetermined temperature. The zirconia material may be composed of various types of material which contain zirconia as a main constituent. Stabilized zirconia or partially stabilized zirconia, in which a part of the zirconia is replaced with a rare earth metal element such as yttria (yttrium oxide) or an alkaline earth metal element, may be used as the zirconia material.

The detection electrode 22 is made of a noble metal material containing Au (gold), which has catalytic activity with respect to ammonia and oxygen, and Pd (palladium) for optimizing the adsorption and oxidation characteristics of Au with respect to ammonia. The noble metal material of the detection electrode 22 can be an Au—Pd alloy, or can contain Au and Pd. The reference electrode 23 is made of a noble metal material such as Pt (platinum) having catalytic activity with respect to oxygen. It would be equally possible for the detection electrode 22 and the reference electrode 23 to contain a zirconia material which becomes a co-material when the detection electrode 22 and the reference electrode 23 are sintered with the first solid electrolyte body 21.

The first surface 211 of the first solid electrolyte body 21, which is exposed to the detection target gas G, forms the outermost surface of the sensor element 10 of the ammonia sensor 1. The detection electrode 22 provided on the first surface 211 is configured such that it can readily come into contact with the detection target gas G. The surface of the detection electrode 22 of this embodiment is not provided with a protective layer made of a porous ceramic material or the like. The detection target gas G thus comes into contact with the detection electrode 22 without being diffusion-controlled. However, it would be possible to provide a protective layer on the surface of the detection electrode 22, if the lowering of the flow velocity of the detection target gas G due to the protective layer is made as small as possible.

The reference electrode 23 provided on the second surface 212 of the first solid electrolyte body 21 is exposed to atmospheric air, as the reference gas A. A reference gas duct (atmospheric air duct) 24 into which the atmospheric air is introduced is formed adjacent to the second surface 212 of the first solid electrolyte body 21.

### (Potential Difference Detection Section 51 and Potential Difference ΔV)

As shown in FIG. 1, the potential difference detection section 51 of the present embodiment detects the potential difference ΔV that arises between the detection electrode 22 and the reference electrode 23 when a hybrid potential is generated at the detection electrode 22. When ammonia and oxygen are present in the detection target gas G that is in contact with the detection electrode 22, the oxidation reaction of ammonia and the reduction reaction of oxygen proceed concurrently at the detection electrode 22. The oxidation reaction of ammonia is typically expressed as: 2NH₃+30²⁻→N₂+3H₂O+6e⁻. The oxygen reduction reaction is typically expressed as: O₂+4e⁻→2O²⁻. A hybrid potential of ammonia and oxygen is generated in the detection electrode 22 when the ammonia oxidation reaction (rate) and the oxygen reduction reaction (rate) at the detection electrode 22 become equal.

FIG. 7 is an explanatory diagram showing a hybrid potential generated at the detection electrode 22. FIG. 7 is a diagram for explaining how the hybrid potential changes, with the horizontal axis expressing values of the potential difference (ΔV) between the detection electrode 22 with respect to the reference electrode 23, and the vertical axis expressing values of current that flows between the detection electrode 22 and the reference electrode 23. In FIG. 7, the first line L1 shows the relationship between the potential difference and the current when the oxidation reaction of ammonia occurs at the detection electrode 22, and the second line L2 shows the relationship between the potential difference and the current when the reduction reaction of oxygen occurs at the detection electrode 22. The first line L1 and the second line L2 are shown as both rising toward the right.

A potential difference ΔV of 0 (zero) indicates that the detection electrode 22 and the reference electrode 23 are at the same potential. The hybrid potential is the value of the potential difference when the current expressed by the first line L1, which is a positive current indicating the ammonia oxidation reaction, is balanced by the current expressed by the second line L2, which is a negative current indicating the oxygen reduction reaction. The hybrid potential is detected at the detection electrode 22 as a negative potential with respect to the reference electrode 23.

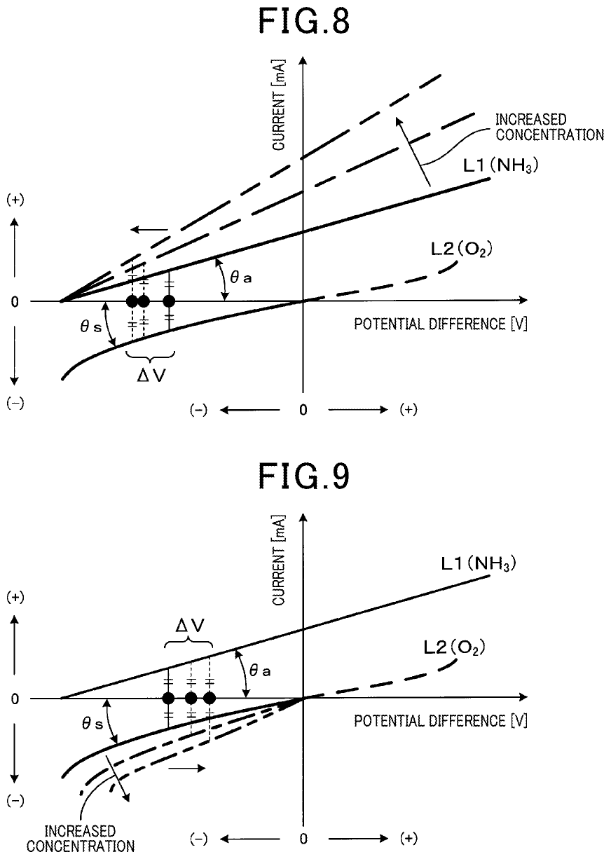

As shown in FIG. 8, when the ammonia concentration in the detection target gas G becomes high, the slope θa of the first line L1, showing the oxidation reaction of ammonia, becomes steep. In this case, the potential at which the positive current on the first line L1 and the negative current on the second line L2 become balanced is shifted in the negative direction. As a result, as the ammonia concentration increases, the potential of the detection electrode 22 becomes increasingly negative with respect to the reference electrode 23. In other words, the higher the ammonia concentration, the greater becomes the potential difference (hybrid potential) ΔV between the detection electrode 22 and the reference electrode 23. Hence, since the higher the ammonia concentration, the greater becomes the potential difference ΔV, the ammonia concentration in the detection target gas G can be detected by detecting the potential difference ΔV.

Furthermore, as shown in FIG. 9, when the oxygen concentration in the detection target gas G becomes high, the slope θs of the second line L2, indicating the oxygen reduction reaction, becomes steep. In that case, the potential at which balance occurs between the positive current and the negative current, expressed by the first line L1 and the second line L2, becomes shifted in the negative direction, to a value that is close to zero. As a result, the higher the oxygen concentration, the smaller becomes the negative potential of the detection electrode 22 with respect to the reference electrode 23. In other words, the higher the oxygen concentration, the smaller becomes the potential difference (hybrid potential) ΔV between the detection electrode 22 and the reference electrode 23. Thus, increased accuracy of detecting the ammonia concentration can be achieved, by applying correction to increase the potential difference ΔV or ammonia concentration in accordance with increase of the oxygen concentration.

### (Temperature of Detection Electrode 22 and Potential Difference ΔV)

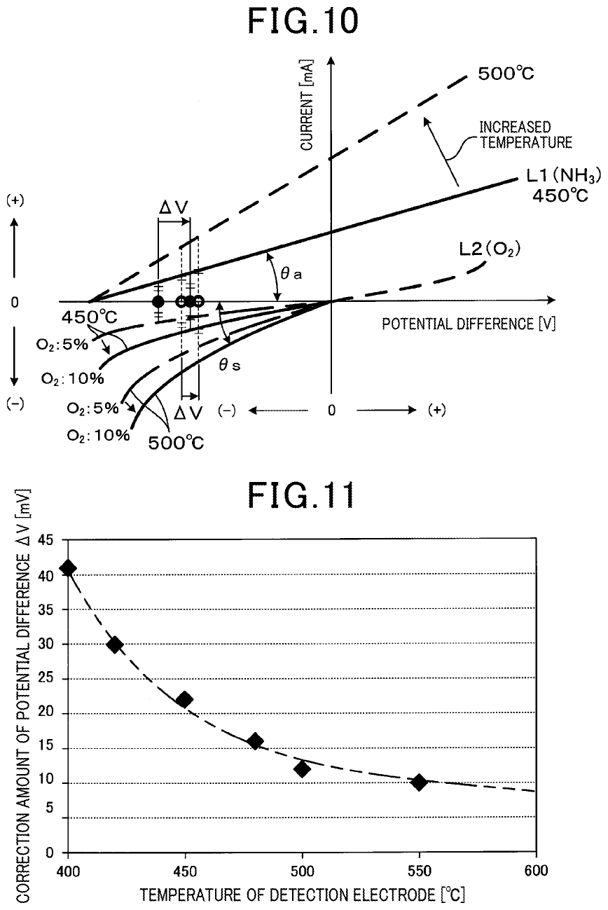

As shown in FIG. 10, when the temperature of the detection electrode 22 (and the ammonia element 2) rises, the slope θa of the first line L1, showing the oxidation reaction of ammonia, becomes steep, while also the slope θs of the second line L2, showing the oxygen reduction reaction, also becomes steep. FIG. 10 shows a case in which the temperature of the detection electrode 22 changes from 450° C. to 500° C. As the temperature of the detection electrode 22 increases, the oxidation current due to the oxidation reaction of ammonia and the reduction current due to the reduction reaction of oxygen respectively increase, and the potential difference (hybrid potential) ΔV decreases. When the temperature of the detection electrode 22 becomes low, these changes occur in the opposite direction.

Furthermore FIG. 10 also shows the change that occurs in the potential difference (hybrid potential) ΔV when the oxygen concentration changes from 5% (volume %) to 10%, for the cases of the temperature of the detection electrode 22 being 450° C. and 500° C., respectively. When the oxygen concentration increases, the potential difference (hybridized potential) ΔV decreases as described above. When the temperature of the detection electrode 22 is 450° C., the amount by which the potential difference (hybrid potential) ΔV becomes reduced when the oxygen concentration changes from 5% to 10% is greater than the amount by which the potential difference becomes reduced when the temperature of the detection electrode 22 is 500° C. and the oxygen concentration changes from 5% to 10%.

In other words, the higher the temperature of the detection electrode 22, the smaller becomes the amount of change in the potential difference (hybrid potential) ΔV when the oxygen concentration changes. Based on this, the higher the temperature of the detection electrode 22, that is, the higher the target control temperature that is set by the energization control section 58, the smaller becomes the amount of correction of the ammonia concentration that is applied by the ammonia concentration calculation section 52 in accordance with an amount of change in the oxygen concentration.

FIG. 11 shows how much the ammonia concentration is corrected by the ammonia concentration calculation section 52 in accordance with an amount of change in the oxygen concentration, when the detection electrode 22 is at a specific temperature between 400 and 600° C. and when the oxygen concentration of the detection target gas G changes from 5% to 10%. The amount of correction of the ammonia concentration is shown as an amount (in millivolts) of correction of the potential difference ΔV. The amount of correction of the potential difference ΔV in this case is an amount applied when the oxygen concentration increases, for increasing the potential difference ΔV.

In FIG. 11, the detection target gas G supplied to the detection electrode 22 was changed from a state of containing 5% (volume %) of oxygen and 100 ppm of ammonia in nitrogen, to a state of containing 10% (volume %) of oxygen and 100 ppm of ammonia in nitrogen. The detection target gas G was supplied to the detection electrode 22 at a flow rate of 500 ml/min. The reference electrode 23 was exposed to atmospheric air.

When the temperature of the detection electrode 22 is as low as about 400° C., the amount of correction of the potential difference ΔV (or of the ammonia concentration) that is applied when the oxygen concentration changes by a specific amount becomes relatively large. On the other hand, when the temperature of the detection electrode 22 is as high as about 550° C., the amount of correction of the potential difference ΔV (or of the ammonia concentration) that is applied when the oxygen concentration changes by a specific amount becomes relatively small. Since the potential difference ΔV indicates the ammonia concentration, correcting the potential difference ΔV is equivalent to correcting the ammonia concentration.

In the ammonia sensor 1 of this embodiment, the energization control section 58 controls the temperature of the detection electrode 22 to be within the range of 400 to 600° C. Since the detection electrode 22 is within the temperature range of 400 to 600° C., the accuracy of calculating the ammonia concentration can be improved by applying correction in accordance with the oxygen. In other words, it has been found by the present inventor that, when using the hybrid potential type ammonia sensor 1, which obtains the ammonia concentration by applying correction in accordance with the oxygen concentration, it is essential for the temperature of the detection electrode 22 to be within the range of 400 to 600° C.

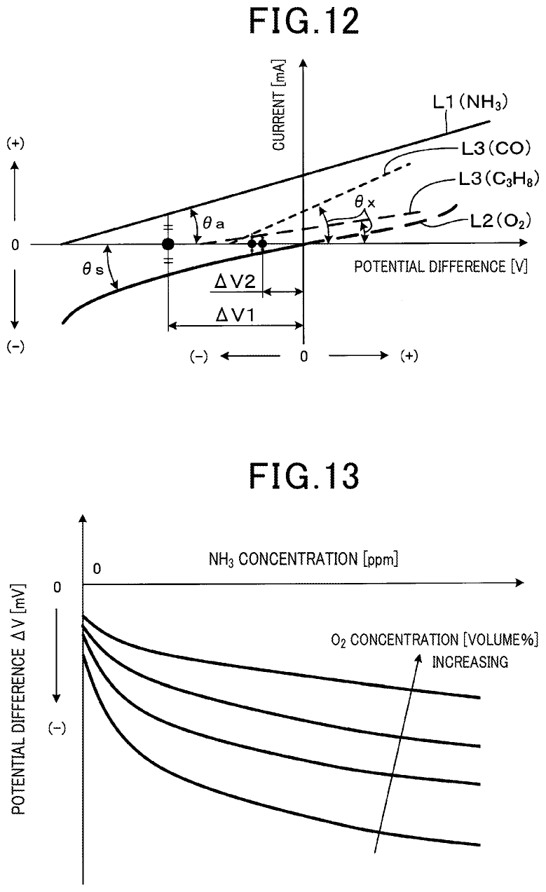

FIG. 12 shows the effect on the potential difference (hybrid potential) ΔV when gases other than ammonia and oxygen, for example, CO, NO, and hydrocarbons (C₃H₈ and the like), are present in the detection target gas G. FIG. 12 shows a case where the other gases are CO and C₃H₈. In FIG. 12, when oxygen, CO and C₃H₈ are present in the detection target gas G, a negative current of the second line L2, which expresses the oxygen reduction reaction, is balanced by a positive current of the first line L1, which expresses the oxidation reaction of ammonia as well as a negative current of the second line L3, which expresses reduction reactions of the other gases CO and C₃H₈.

Since the negative potentials of CO and C₃H₈ are smaller than the negative potential of ammonia, the hybrid potential ΔV2, at which the reduction reaction of oxygen is in balance with the oxidation reaction of the CO or C₃H₈, is lower than the hybrid potential ΔV1 at which the reduction reaction of oxygen is in balance with the oxidation reaction of ammonia (where ΔV2 is a negative potential close to zero). As a result, the hybrid potential ΔV1 that indicates the ammonia concentration is affected by the hybrid potential ΔV2 that indicates the concentration of the other gas(es), and hence the accuracy of detecting the hybrid potential ΔV1 may be lowered. In other words, there is a danger that the hybrid potential ΔV1 may become combined with the hybrid potential ΔV2. Furthermore, the temperature dependencies of the hybrid potential ΔV1 and the hybrid potential ΔV2 will be different from one another.

When the temperature of the detection electrode 22 becomes lower, in FIG. 12, the slope θa of the first line L1, showing the oxidation reaction of ammonia, the slope θs of the second line L2 showing the reduction reaction of oxygen, and the slope(s) θx of the third line(s) L3 showing the oxidation reaction of another gas(es) become respectively smaller, and the potential difference (hybrid potential) ΔV1 indicating the ammonia concentration becomes more susceptible to the effects of the other gases.

When the temperature of the detection electrode 22 is 400° C. or higher, the oxidation catalyst performance of the detection electrode 22 with respect to ammonia is significantly higher than the oxidation catalyst performance of the detection electrode 22 with respect to other gases. Hence the hybrid potential ΔV1 resulting from the oxidation reaction of ammonia and the reduction reaction of oxygen is not significantly affected by the hybrid potential ΔV2 that results from the oxidation reaction of other gases and the reduction reaction of oxygen.

On the other hand, if the temperature of the detection electrode 22 is less than 400° C., the difference between the oxidation catalyst performance of the detection electrode 22 with respect to ammonia and the oxidation catalyst performance of the detection electrode 22 with respect to other gases becomes small. Hence, the hybrid potential ΔV1 resulting from the oxidation reaction of ammonia and the reduction reaction of oxygen is readily affected by the hybrid potential ΔV2 that results from the oxidation reaction of other gases and the reduction reaction of oxygen.

When the temperature of the detection electrode 22 exceeds 600° C., the slope θa of the first line L1 showing the oxidation reaction of ammonia and the slope θs of the second line L2 showing the reduction reaction of oxygen become substantially steep. In that case the potential difference ΔV, at which the positive current value expressing the oxidation reaction of ammonia and the negative current value expressing the oxygen reduction reaction become balanced, will tend to approach the zero origin. Thus, the absolute value of the hybrid potential ΔV1 or the detected ammonia concentration becomes small so that the accuracy of detecting the ammonia concentration decreases.

Hence, by controlling the temperature of the detection electrode 22 to be within the range of 400 to 600° C. by means of the energization control section 58, high accuracy of detecting the ammonia concentration after oxygen correction can be maintained. It has been confirmed that when 10 ppm or more, for example, of ammonia is contained in exhaust gas as the detection target gas G, other gases such as NOx, CO, and HC (hydrocarbons), which may be contained in the gas G, do not significantly affect the accuracy of detecting the ammonia concentration if the temperature of the detection electrode 22 is within the range of 400 to 600° C.

### (Oxygen Element Section 3)

As shown in FIGS. 1 and 5, the ammonia sensor 1 of the present embodiment includes an oxygen element section 3, a pumping section 53, a pump current detection section 54, an oxygen concentration calculation section 55, a NOx detection section 56, and a NOx concentration calculation section 57, in addition to an ammonia element section 2, a potential difference detection section 51, an ammonia concentration calculation section 52, a heater section 4, and an energization control section 58. The oxygen element section 3 is stacked with a heater section 4, for heating the oxygen element section 3 and the ammonia element section 2 to configure a multi-gas sensor.

The oxygen element section 3 has a second solid electrolyte body 31, a gas chamber 35, a diffusion resistance section 351, a pump electrode 32, a NOx electrode 33, and other reference electrodes 34. The second solid electrolyte body 31 is disposed facing the first solid electrolyte body 21. The second solid electrolyte body 31 is formed in a plate shape, and is made of a zirconia material having a property of conducting oxygen ions when the material is at a predetermined temperature. This zirconia material is the same as that of the first solid electrolyte body 21.

It should be noted that if the ammonia sensor 1 is not required to have a NOx detection function, then the oxygen element section 3 is not required to have the NOx electrode 33, and the ammonia sensor 1 is not required to have the NOx detection section 56 and the NOx concentration calculation section 57.

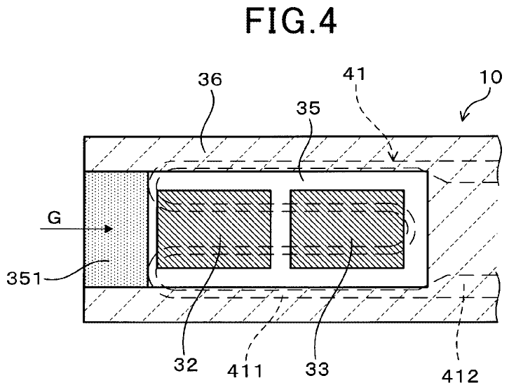

As shown in FIGS. 1, 2 and 4, the gas chamber 35 is formed adjoining the third surface 311 of the second solid electrolyte body 31. The gas chamber 35 is formed in a gas chamber insulator 36. The gas chamber insulator 36 is made of a ceramic material such as alumina. The diffusion resistance section 351 is formed as a porous ceramic layer, and serves to introduce the detection target gas G into the gas chamber 35 while limiting the diffusion rate of the gas.

The pump electrode 32 is housed in the gas chamber 35 on the third surface 311, exposed to the detection target gas G in the gas chamber 35. The NOx electrode 33 is housed in the gas chamber 35 on the third surface 311, and comes in contact with the detection target gas G after the oxygen concentration of the gas G has been adjusted by the pump electrode 32. The other reference electrodes 34 are provided on the fourth surface 312 of the second solid electrolyte body 31, which is disposed opposite the third surface 311 of the second solid electrolyte body 31.

The pump electrode 32 is made of a noble metal material that has catalytic activity for oxygen but does not have catalytic activity for NOx. The noble metal material of the pump electrode 32 can be composed of a Pt—Au alloy or a material containing Pt and Au. The NOx electrode 33 is formed using a noble metal material having catalytic activity for NOx and oxygen. The noble metal material of the NOx electrode 33 can be composed of a Pt—Rh (rhodium) alloy or a material containing Pt and Rh. The other reference electrodes 34 are formed using a noble metal material such as Pt which has catalytic activity for oxygen. Furthermore, it would be possible for the pump electrode 32, the NOx electrode 33 and the other reference electrodes 34 to contain a zirconia material which becomes a co-material when the reference electrodes 34 are sintered with the second solid electrolyte body 31.

One of the other reference electrodes 34 of this embodiment is provided at a position facing the pump electrode 32, opposite the NOx electrode 33 via the second solid electrolyte body 31. It should be noted that it would be possible to use a single reference electrode 34, disposed opposite all of the pump electrode 32 and the NOx electrode 33.

As shown in FIGS. 1 to 3, the other reference electrodes 34 provided on the fourth surface 312 of the second solid electrolyte body 31 are exposed to atmospheric air as the reference gas A. The first solid electrolyte body 21 and the second solid electrolyte body 31 are stacked together via a duct insulator 25, in which a reference gas duct 24 is formed. The duct insulator 25 is made of a ceramic material such as alumina.

The reference gas duct 24 is formed such that the reference electrode 23 on the second surface 212 of the first solid electrolyte body 21 and the other reference electrodes 34 on the fourth surface 312 of the second solid electrolyte body 31 are exposed to atmospheric air. The reference electrode 23 and the other reference electrodes 34 are housed within the reference gas duct 24. The reference gas duct 24 is formed such as to extend from the base end of the sensor element 10 to a position facing the gas chamber 35.

Reference gas A that is introduced into a base end cover of the ammonia sensor 1 passes into the reference gas duct 24 from an opening at the base end of the reference gas duct 24. With the sensor element 10 of the present embodiment, the reference gas duct 24 is located between the first solid electrolyte body 21 and the second solid electrolyte body 31, so that all of the reference electrode 23 and the other reference electrodes 34 can be exposed to the atmospheric air.

### (Pumping Section 53, Pump Current Detection Section 54 and Oxygen Concentration Calculation Section 55)

The pumping section 53 shown in FIG. 1 is configured to pump oxygen from the detection target gas G in the gas chamber 35 by applying a DC voltage between the pump electrode 32 and one of the other reference electrodes 34, with that other reference electrode 34 as the positive polarity electrode. When the DC voltage is applied between the pump electrode 32 and the other reference electrode 34, the oxygen in the detection target gas G in the gas chamber 35 that comes into contact with the pump electrode 32 becomes ionized, flows through the second solid electrolyte body 31 to the other reference electrode 34, and is discharged from the reference electrode 23 into the reference gas duct 24. The oxygen concentration in the gas chamber 35 is thereby adjusted to a value that is suitable for detecting NOx.

The pump current detection section 54 is configured to detect a DC current that flows between the pump electrode 32 and the other reference electrode 34. The oxygen concentration calculation section 55 is configured to calculate the oxygen concentration in the detection target gas G based on the DC current detected by the pump current detection section 54. The pump current detection section 54 detects a DC current that is proportional to the rate of discharge of oxygen from the gas chamber 35 into the reference gas duct 24 by the pumping section 53.

The pumping section 53 discharges oxygen from the gas chamber 35 into the reference gas duct 24 until the oxygen concentration in the detection target gas G in the gas chamber 35 reaches a prescribed value.

Thus, by monitoring the DC current detected by the pump current detection section 54, the oxygen concentration calculation section 55 can calculate the oxygen concentration in the detection target gas G that reaches the ammonia element section 2 and the oxygen element section 3.

The oxygen concentration calculated by the oxygen concentration calculation section 55 is used as the oxygen concentration for correcting the ammonia concentration that is obtained by the ammonia concentration calculation section 52.

### (NOx Detection Section 56 and NOx Concentration Calculation Section 57)

As shown in FIG. 1, the NOx detection section 56 is configured to apply a DC voltage between the NOx electrode 33 and the other reference electrode 34, with the other reference electrode 34 as the positive polarity electrode, and to detect a DC current flowing between the NOx electrode 33 and the other reference electrode 34. The NOx concentration calculation section 57 is configured to calculate the pre-correction NOx concentration in the detection target gas G based on the DC current detected by the NOx detection section 56, and to subtract the ammonia concentration obtained by the ammonia concentration calculation section 52 from the pre-correction NOx concentration, to obtain the corrected NOx concentration. The NOx detection section 56 detects not only NOx but also ammonia. Hence, the NOx concentration calculation section 57 can obtain the actual detected amount of NOx by subtracting the detected amount of ammonia.

Two types of NOx concentration are obtained by the NOx concentration calculation section 57. The NOx concentration that is obtained based on the current generated in the NOx detection section 56 is designated as the pre-correction NOx concentration. The pre-correction NOx concentration includes an ammonia concentration that is due to ammonia which reacts at the NOx electrode 33. On the other hand, the concentration obtained by subtracting the ammonia concentration obtained by the ammonia concentration calculation section 52 from the pre-correction NOx concentration obtained by the NOx concentration calculation section 57 is designated as the corrected NOx concentration. The corrected NOx concentration indicates the NOx concentration with the effects of ammonia excluded. When the ammonia concentration and the NOx concentration are compared, the corrected NOx concentration is used.

The NOx electrode 33 comes into contact with the detection target gas G after the oxygen concentration in the detection target gas G has been adjusted by the pump electrode 32. When a DC voltage is applied by the NOx detection section 56 between the NOx electrode 33 and the other reference electrode 34, the NOx in contact with the NOx electrode 33 is decomposed into nitrogen and oxygen, the oxygen becomes oxygen ions, which pass through the second solid electrolyte body 31 to the other reference electrode 34, and are then discharged from the reference electrode 23 into the reference gas duct 24. Furthermore, when ammonia reaches the NOx detection section 56, NOx produced by oxidizing the ammonia is decomposed into nitrogen and oxygen in the same way. The NOx concentration calculation section 57 calculates the pre-correction NOx concentration in the detection target gas G that reaches the oxygen element section 3 by monitoring the DC current detected by the NOx detection section 56, and calculate NOx concentration as the corrected NOx concentration by subtracting the ammonia concentration from the pre-correction NOx concentration.

By making the ammonia sensor 1 a multi-gas sensor that detects not only the ammonia concentration but also the oxygen concentration and the NOx concentration, the number of gas sensors used in the exhaust pipe 71 for detecting the ammonia concentration and the NOx concentration can be reduced. Furthermore, the oxygen concentration can be detected by the pump current detection section 54 and the oxygen concentration calculation section 55 through use of the same pump electrode 32 and pumping section 53 that are used for detecting the NOx concentration.

The pumping section 53, the pump current detection section 54, and the NOx detection section 56 are configured using amplifiers, etc. in the sensor control unit 5. The oxygen concentration calculation section 55 and the NOx concentration calculation section 57 are configured using computers or the like in the sensor control unit 5.

It should be noted that, in FIG. 1, for convenience, the potential difference detection section 51, the pumping section 53, the pump current detection section 54, and the NOx detection section 56 are shown as being separate from the sensor control unit 5. However, in actuality they are built into the sensor control unit 5. Although not shown, the electrical connecting leads of the electrodes 22, 23, 32, 33, and 34 are formed extending to the base end of the sensor element 10, as for the lead 412 of the heating body 41.

### (Ammonia Concentration Calculation Section 52)

As shown in FIGS. 1 and 5, the ammonia concentration calculation section 52 calculates the ammonia concentration in the detection target gas G based on the oxygen concentration obtained by the oxygen concentration calculation section 55 and the potential difference ΔV obtained by the potential difference detection section 51. The sensor control unit 5 of the ammonia sensor 1 is configured to obtain the ammonia output concentration by correcting an ammonia concentration that is obtained based on the potential difference ΔV detected by the potential difference detection section 51, with the correction of the ammonia concentration being performed using an oxygen concentration that is obtained based on the DC current detected by the pump current detection section 54, while together with this, the sensor control unit 5 is configured to obtain the NOx concentration based on the DC current detected by the NOx detection section 56.

FIG. 13 shows how the potential difference (hybrid potential) ΔV between the detection electrode 22 and the reference electrode 23 obtained by the potential difference detection section 51, which is detected by the hybrid potential type ammonia element section 2 based on variations in the ammonia concentration in the detection target gas G, changes under the effects of the oxygen concentration. As shown in FIG. 13, the detected potential difference (hybrid potential) ΔV detected by the potential difference detection section 51 becomes smaller (is detected as a negative potential that approaches zero) as the oxygen concentration increases. The reason for this has been described above, based on the slope θs in FIG. 9.

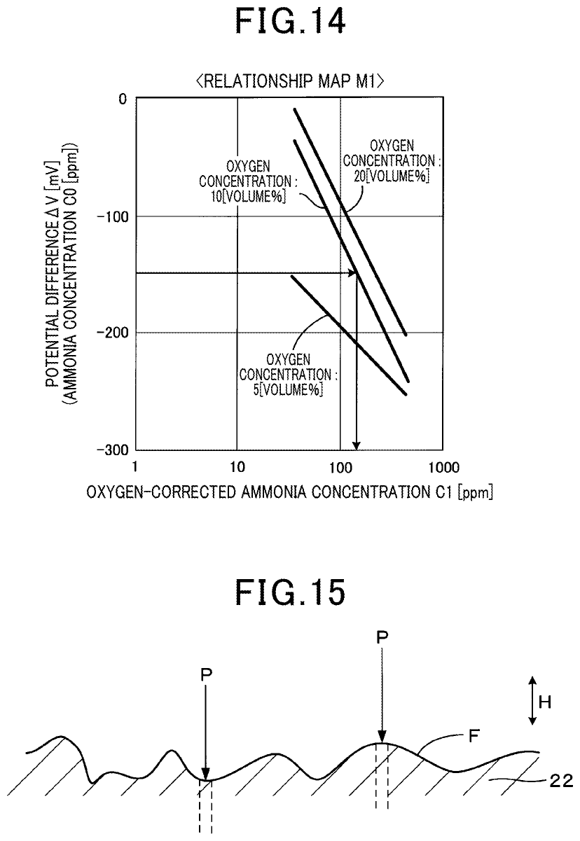

A relationship map M1 as shown in FIG. 14 is set in the ammonia concentration calculation section 52 of the present embodiment, with the map having the oxygen concentration in the detection target gas G as a parameter, and expressing the relationship between the potential difference ΔV obtained by the potential difference detection section 51 and the corrected ammonia concentration C1 which has been corrected in accordance with the oxygen concentration. The relationship map M1 is created as expressing the respective relationships, for each of prescribed oxygen concentrations, between the potential difference ΔV (indicating the pre-correction ammonia concentration C0) and the corrected values of ammonia concentration C1. The ammonia concentration calculation section 52 is configured to use the relationship map M1 to calculate the oxygen-corrected ammonia concentration C1 in the detection target gas G, by collating the oxygen concentration in the detection target gas G with the potential difference ΔV obtained by the potential difference detection section 51.

More specifically, the ammonia concentration calculation section 52 collates the oxygen concentration obtained by the oxygen concentration calculation section 55 and the potential difference ΔV obtained by the potential difference detection section 51 with the oxygen concentration values and the potential difference ΔV values of the relationship map M1. The corrected ammonia concentration C1 that corresponds to the obtained potential difference ΔV is then read out from the relationship map M1. The ammonia concentration calculation section 52 then applies correction such that the higher the oxygen concentration, the higher becomes the corrected ammonia concentration C1. The oxygen-corrected ammonia concentration C1, amended in accordance with the oxygen concentration, becomes the ammonia output concentration that is outputted from the ammonia sensor 1 as shown in FIG. 5. It should be noted that it would be equally possible for pre-correction values of ammonia concentration CO to replace the potential difference ΔV values in the relationship map M1.

FIG. 14 shows an example of the relationship map M1 for oxygen concentrations of 5 [volume %], 10 [volume %], and 20 [volume %] in the detection target gas G. By using this relationship map M1, the potential difference ΔV (or ammonia concentration CO) can readily be corrected in accordance with the oxygen concentration. The relationship map M1, relating values of the potential difference ΔV to corrected values of oxygen concentration C1, may be derived at the time of production trials or testing, etc., of the ammonia sensor 1.

Furthermore, relationship maps M1 as shown in FIG. 14 may be established for each of respective temperatures of the detection electrode 22.

The oxygen-corrected ammonia concentration C1, corrected in accordance with the oxygen concentration, can thereby be calculated taking into account differences in the temperature of the detection electrode 22. Alternatively, the oxygen-corrected ammonia concentration C1 that has been calculated from the relationship map M1 could then be corrected based on the temperature of the detection electrode 22, by using a predetermined temperature correction coefficient.

The potential difference detection section 51 and the ammonia concentration calculation section 52 are constituted in the sensor control unit (SCU) 5, which is electrically connected to the ammonia sensor 1. The potential difference detection section 51 is formed using amplifiers, etc., which measure the potential difference ΔV between the detection electrode 22 and the reference electrode 23. The ammonia concentration calculation section 52 is formed using a computer or the like. The sensor control unit 5 is furthermore connected to the engine control unit (ECU) 50 of the internal combustion engine 7, and is used by the engine control unit 50 in controlling the operation of the internal combustion engine 7 and of the reducing agent supply device 73, etc.

It should be noted that, when correcting the ammonia concentration in accordance with the oxygen concentration, the ammonia concentration calculation section 52 could perform that correction taking into account the pre-correction NOx concentration or the corrected NOx concentration which are obtained by the NOx detection section 56. The NOx electrode 33 in the oxygen element section 3 not only has a catalytic activity for NOx, but also for ammonia. Hence, the ammonia concentration can be detected as the pre-correction NOx concentration at the NOx electrode 33. As a result, the ammonia concentration expressed by the potential difference ΔV can be corrected by the ammonia concentration calculation section 52 based on the oxygen concentration, the temperature of the detection electrode 22, and the NOx concentration.

### (Heater Section 4 and Energization Control Section 58)

As shown in FIGS. 1 and 2, the heater section 4, which heats the oxygen element section 3 and the ammonia element section 2, is stacked on the side of the second solid electrolyte body 31 opposite the side on which the first solid electrolyte body 21 is stacked. In other words, the heater section 4 is stacked on the side of the oxygen element section 3 that is opposite the side on which the ammonia element section 2 is stacked.

The heater section 4 is formed of a heating body 41 that generates heat when energized and a heater insulator 42 in which the heating body 41 is embedded. The heater insulator 42 is made of a ceramic material such as alumina. The reference gas duct 24 into which the reference gas A is introduced is formed between the ammonia element section 2 and the oxygen element section 3. The reference electrode 23 and the other reference electrodes 34 are housed within the reference gas duct 24.

As shown in FIGS. 1 to 4, the heating body 41 is formed with a heat generating section 411 and a lead section 412 to which the heat generation section 411 is connected, and the heat generating section 411 is formed opposite the electrodes 22, 23, 32, 33, 34 with respect to the stacking direction (hereinafter referred to as the stacking direction H) of the solid electrolyte bodies 21, 31 and the insulators 25, 36, 42. An energization control section 58 for energizing the heating body 41 is connected to the heating body 41. The degree of energization of the heating body 41 by the energization control section 58 is adjusted by changing the voltage applied to the heating body 41. The energization control section 58 is formed using a drive circuit or the like which applies a voltage subjected to PWM (pulse width modulation) control or the like to the heating body 41. The energization control section 58 is constituted within the sensor control unit 5.

The distance between the ammonia element section 2 and the heater section 4 is greater than the distance between the oxygen element section 3 and the heater section 4. The temperature to which the ammonia element 2 is heated by the heater section 4 is lower than the temperature to which the oxygen element section 3 is heated by the heater section 4. The pump electrode 32 and NOx electrode 33 of the oxygen element section 3 are used within an operating temperature range of 600 to 900° C., and the detection electrode 22 of the ammonia element section 2 is used within an operating temperature range of 400 to 600° C. The lower limit operating temperature of the detection electrode 22 is 400° C., and the upper limit operating temperature is 600° C.

The temperature of the detection electrode 22 is controlled to a target temperature, which can have any value within the operating temperature range of 400 to 600° C., through heating by the heater section 4. The energization control section 58 is configured such that when the temperature of the detection electrode 22 is controlled to the target control temperature, the NOx electrode 33 is heated to a value within the operating temperature range of 600 to 900° C. With this configuration, the heating control of the heater section 4 by the energization control section 58 can bring the detection electrode 22 of the ammonia element section 2 to a temperature suitable for ammonia detection, and bring the NOx electrode 33 of the oxygen element section 3 to a temperature suitable for oxygen detection.

Furthermore, since the reference gas duct 24 is formed between the oxygen element section 3 and the ammonia element section 2, the reference gas duct 24 can act as an insulating layer when the heater section 4 heats the oxygen element section 3 and the ammonia element section 2. As a result, the temperature of the detection electrode 22 of the ammonia element section 2 can readily be make lower than the temperature of the pump electrode 32 and the NOx electrode 33 of the oxygen element section 3. Furthermore, the temperatures of the oxygen element section 3 and the ammonia element section 2 are controlled to target values by the energization control that is executed by the energization control section 58.

### (Composition of Detection Electrode 22)

By setting the content ratio of Au and Pd in the noble metal material of the detection electrode 22 of this embodiment within a suitable range, the sensitivity to ammonia due to the Au is appropriately weakened, and there is a reduced likelihood of detection errors in the sensor output of the detection electrode 22. The content ratio of Au and Pd is specified for the surface of the detection electrode 22, where the decomposition reaction and the oxidation reaction of ammonia occur. The content ratio of Au and Pd can be defined as the ratio of the respective proportions of Au exposed on the surface of the detection electrode 22 and Pd exposed on the surface of the detection electrode 22. Furthermore, as shown in FIG. 15, the content ratio of Au and Pd on the surface of the detection electrode 22 can be the ratio of Au and Pd within a depth range of 1 μm from the outermost face F of the detection electrode 22, as measured along the stacking direction H. The stacking direction H is perpendicular to the first surface 211 of the first solid electrolyte body 21.

The detection electrode 22 is fired in a condition in which Au—Pd alloy particles and zirconia particles are mixed. The content ratio of Au and Pd can be the ratio of Au and Pd in Au—Pd alloy particles that are present in the outermost face of the detection electrode 22. Alternatively, the detection electrode 22 may be fired in a condition in which Au particles, Pd particles and zirconia particles are mixed.

The surface of the detection electrode 22 is not formed with a flat shape, but has an uneven shape due to the presence of particles of Au, Pd, and solid electrolyte body. The surface of the detection electrode 22, which determines the content ratio of Au and Pd, is uneven. The 1 μm range of depth from the outermost face F of the detection electrode 22 can be set as being a range of depth from tip portions of the uneven shape of the outermost face F, as measured in the stacking direction H.

On the surface of the detection electrode 22, the molar ratio of Au and Pd is in the range Au:Pd=100:80-100:0.5. This indicates that the proportion of Au and the proportion of Pd on the surface of the detection electrode 22 are in the range Au:Pd=100:80-100:0.5.

### (Measurement Method)

The content ratio of Au and Pd on the surface of the detection electrode 22 can be measured as follows. The content ratio can be measured by using X-ray photoelectron spectroscopy (XPS), for example. In XPS, the surface of a sample is irradiated with X-rays, and the distribution of the kinetic energy of photoelectrons emitted from this surface is measured. The type, abundance, chemical bond state, etc. of the elements that are present within a depth range of about several nm from the surface of the sample are measured. The ESCALAB200 manufactured by Thermo Fisher Scientific Co., Ltd. can be used for XPS, for example.

With this embodiment as shown in FIG. 15, the detection electrode 22 is sliced in the stacking direction H, at a suitable location, and the amounts of Au and Pd present within a depth range extending 1 μm in the stacking direction H from the outermost surface F are measured, with the measurement being executed at a plurality of measurement sites P on the cut surface. The positions of the measurement sites P on the surface of the detection electrode 22 are determined with respect to directions in the plane of the detection electrode 22. Hence, the measurement sites P have appropriately different positions in the stacking direction H relative to the plane of the detection electrode 22.

(Sensor Output Sensitivity S and Variation with Time E)

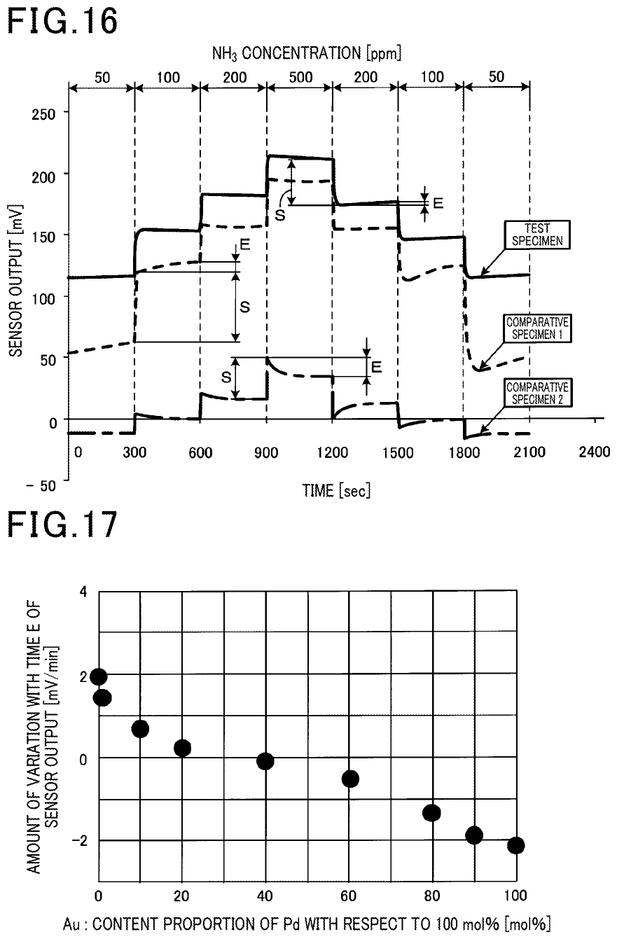

FIG. 16 shows results of the sensor output sensitivity S of the detection electrode 22 when the ammonia concentration in a test gas is varied, and the amount of variation with time E in the sensor output of the detection electrode 22 when the ammonia concentration of the test gas is held constant, for the case in which the noble metal material of the detection electrode 22 contains only Au (comparative specimen 1), the case in which the noble metal material contains Pd: 100 mol % with respect to Au: 100 mol % (comparative specimen 2), and the case in which the noble metal material contains Pd: 10 mol % with respect to Au: 100 mol % (test specimen). The test gas contained ammonia, oxygen and nitrogen. The oxygen concentration in the test gas was 10% by volume, the ammonia concentration in the test gas was varied between 50 and 500 ppm, and the remainder of the test gas was nitrogen.

In FIG. 16, the sensitivity S and amount of variation with time E of the sensor output of the detection electrode 22 are obtained as amounts of change of the potential difference ΔV [mV] between the detection electrode 22 and the reference electrode 23, when the ammonia concentration [ppm] in the test gas was changed in the sequence 50 ppm, 100 ppm, 200 ppm, 500 ppm, 200 ppm, 100 ppm, and 50 ppm, at intervals of 300 seconds (5 minutes).

The sensitivity S of the sensor output of the detection electrode 22 indicates the amount of change that occurs in the sensor output when the ammonia concentration of the test gas (or the detection target gas G) changes. Specifically, the sensitivity S is expressed by the amount of change in the potential difference ΔV between the detection electrode 22 and the reference electrode 23 that occurs when the detection electrode 22 detects a change in the ammonia concentration. That is, the sensitivity S is expressed by the difference (absolute value) between the sensor output at the time point immediately before a change occurs in the ammonia concentration and the sensor output at the time point immediately following the change in the ammonia concentration.

The amount of variation with time E in the sensor output of the detection electrode 22 indicates the amount of change (deviation amount) in the sensor output during a condition in which the ammonia concentration of the test gas (or the detection target gas G) is held constant immediately after the ammonia concentration has changed. The amount of variation with time E indicates an amount of deviation of the potential difference ΔV between the detection electrode 22 and the reference electrode 23 that occurs after the detection electrode 22 detects a change in ammonia concentration. The amount of variation with time E in the sensor output is expressed by the difference (absolute value) between the sensor output at the time point immediately after the ammonia concentration changes and the sensor output at the time point when the ammonia concentration subsequently changes again.

In FIG. 16, the absolute magnitude of the sensor output of the detection electrode 22 for the comparative specimens 1 and 2 and for the test specimen is detected in a state of offset from the zero point at which the sensor output is 0 mV. This offset amount is corrected and adjusted by a control device of the ammonia sensor 1.

It can be seen that with comparative specimen 1, in which the noble metal material of the detection electrode 22 consists only of Au, the sensitivity S of the sensor output is high, showing that the sensitivity of the detection electrode 22 to ammonia is high. However, in the case of comparative specimen 1 it can be seen that the amount of variation E of the sensor output with time is also large, so that detection errors are liable to occur in the sensor output.

It can be seen that in comparative specimen 2 in which the noble metal material of the detection electrode 22 contains Pd: 100 mol % with respect to Au: 100 mol %, the sensitivity S of the sensor output is low, showing that the sensitivity of the detection electrode 22 to ammonia is low. Furthermore, it can be seen that in the case of the comparative specimen 2, the amount of variation E of the sensor output with time is large, so that detection errors are liable to occur in the sensor output.

On the other hand, in the case of the test specimen in which the noble metal material of the detection electrode 22 contains Pd: 10 mol % with respect to Au: 100 mol %, it can be seen that the sensitivity S of the sensor output is relatively high, and the sensitivity of the detection electrode 22 to ammonia is moderately high. Furthermore, it can be seen that with this test specimen, the amount of variation E of the sensor output with time is small, so that detection errors are not liable to occur in the sensor output.

FIG. 17 shows the amount of variation with time E (mV/min) of the sensor output of the detection electrode 22 when the content proportion of Pd with respect to Au: 100 mol % on the surface of the detection electrode 22 is varied in the range 0 to 100 mol %. The composition of the test gas used in this measurement is the same as in the case of FIG. 16. FIG. 17 shows the amount of variation with time E of the sensor output of the detection electrode 22 in a condition in which the ammonia concentration of the test gas was held at 100 ppm after being changed from 50 ppm to 100 ppm. When Pd is 0 mol %, this indicates that the noble metal material of the detection electrode 22 consists only of Au.

It can be seen from FIG. 17 that when the content proportion of Pd with respect to Au: 100 mol % is 20 to 40 mol %, the amount of variation with time E is close to 0 mV/min, and so can be held at a small value. On the other hand, it can be seen that when the content proportion of Pd with respect to Au is less than 20 mol %, and when the content proportion of Pd with respect to Au exceeds 40 mol %, the amount of variation with time E is large.

In particular, when the content proportion of Pd with respect to Au is in the region of 30 mol %, the amount of variation with time E increases in the positive direction when the content proportion of Pd decreases, whereas when the content proportion of Pd increases, the amount of variation with time E increases in the negative direction. When the Pd content proportion is less than 20 mol %, then since the Au content proportion is increased, the adsorption characteristic due to the Au becomes higher, and so the amount of variation with time E increases in the positive direction. On the other hand, when the Pd content proportion exceeds 40 mol %, then since the Pd content proportion is increased, the oxidation characteristic due to the Pd become higher, and so the amount of variation with time E increases in the negative direction.

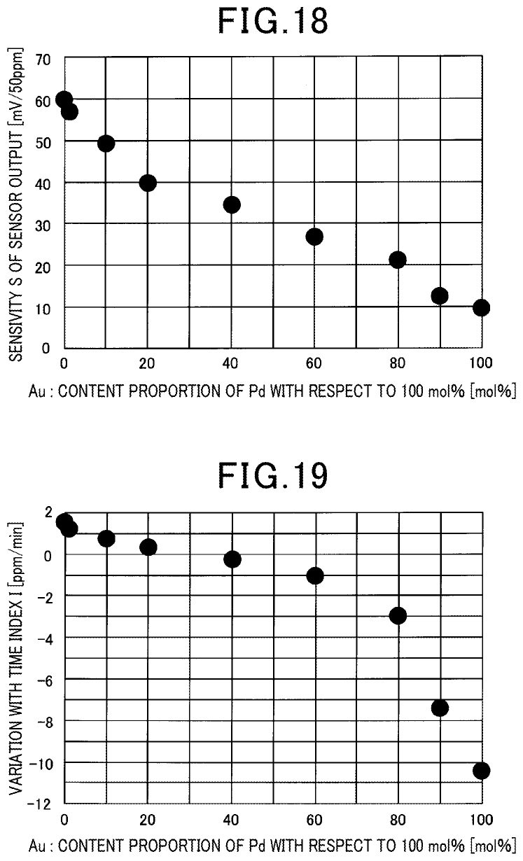

FIG. 18 shows the results of measuring the sensitivity S [mV/50 ppm] of the sensor output of the detection electrode 22 when the content proportion of Pd with respect to Au: 100 mol % on the surface of the detection electrode 22 is varied within the range of 0 to 100 mol %. The composition of the test gas used in this measurement is the same as in the case of FIG. 16. FIG. 18 shows the sensitivity S, which is the amount of change in the sensor output of the detection electrode 22 that occurs when the ammonia concentration of the test gas changes from 50 ppm to 100 ppm.

It can be seen from FIG. 18 that the sensitivity S of the sensor output of the detection electrode 22 increases as the content proportion of Pd with respect to Au: 100 mol % decreases from 100 mol % to 0 mol %. In particular it can be seen that when the content proportion of Pd with respect to Au: 100 mol % is 20 mol % or less, the sensor output sensitivity S of the detection electrode 22 to ammonia is high.

The content proportion of Pd with respect to Au: 100 mol % on the surface of the detection electrode 22 is selected to be within a range in which the sensitivity of the detection electrode 22 to ammonia is as high as possible and the variation with time of the sensor output of the detection electrode 22 is as small as possible. The content proportion of Pd with respect to Au: 100 mol % on the surface of the detection electrode 22 is determined based on the variation-with-time index I [ppm/min]), obtained by dividing the variation with time E (mV/min) of the sensor output by the sensor output sensitivity S [mv/ppm). The variation-with-time index I is obtained as I=E/S×50 [ppm], by converting to the amount of potential change per 1 ppm of ammonia concentration.

The variation-with-time index I expresses the extent to which the sensor output fluctuates (deviates) after the ammonia concentration changes, and the magnitude of the sensor output sensitivity (change amount) in response to a change in the ammonia concentration. The closer the variation-with-time index approaches 0 ppm/min, the less likely become fluctuations (deviations) of the sensor output.

FIG. 19 summarizes the measurement results of FIGS. 17 and 18 and shows values of the variation-with-time index I obtained for respective Pd content proportions with respect to Au: 100 mol %. As can be seen in FIG. 19, when the content proportion of Pd with respect to 100 mol % of Au exceeds 80 mol %, the variation-with-time index I deteriorates abruptly. Hence, by setting the content proportion of Pd to be within 80 mol % or less with respect to Au: 100 mol %, it is possible to appropriately suppress variation with time of the sensor output, while appropriately maintaining the sensitivity of the sensor output to ammonia.

Furthermore, if the content proportion of Pd to Au: 100 mol % is 0.5 mol % or more, 40 mol % or less, the sensitivity of the sensor output to ammonia can be maintained, and variation with time of the sensor output can be suppressed more effectively. Furthermore, when the content proportion of Pd with respect to Au: 100 mol % is 20 to 40 mol %, the variation-with-time index I becomes close to 0 ppm/min, and this content proportion is the most appropriate from the aspects of maintaining the sensor output sensitivity to ammonia and of suppressing variation with time of the sensor output.

Au on the surface of the detection electrode 22 has a high adsorption characteristic and high sensitivity to ammonia. However, the high adsorption characteristic of the Au causes the variation-with-time index I in FIG. 19 to becomes increased in the positive direction. On the other hand, Pd on the surface of the detection electrode 22 has a high oxidizing characteristic and low sensitivity to ammonia. However, the high oxidizing characteristic of the Pd causes the variation-with-time index I in FIG. 19 to become increased in the negative direction. Hence, it can be understood that it is important to establish an appropriate balance between the adsorption property of Au and the oxidation property of Pd on the surface of the detection electrode 22, in order to bring the value of the variation-with-time index I close to 0 ppm/min.

### (Operational Effects)

The ammonia sensor 1 of this embodiment is of hybrid potential type, detecting attainment of a potential difference ΔV, which is the potential difference value that arises when the oxygen reduction reaction and the ammonia oxidation reaction are balanced. As a result of thorough research by the present inventor, it has been found that by setting an appropriate content ratio of Au and Pd in the noble metal of the detection electrode 22, an appropriate balance can be maintained between the adsorption property and the oxidation property of the detection electrode 22 with respect to ammonia, and variation with time of the sensor output of the detection electrode 22 can be appropriately suppressed.

If the content proportion of Pd with respect to Au on the surface of the detection electrode 22 is excessively high, then the oxidation characteristic of the detection electrode 22 become high, and not only does the sensitivity of the detection electrode 22 decrease, but also the variation with time of the sensor output of the detection electrode 22 becomes increased in the negative direction. If the content proportion of Pd with respect to Au in the detection electrode 22 is more than 0 mol % and 80 mol % or less, then variation with time of the sensor output of the detection electrode 22 can be appropriately suppressed, while maintaining an appropriate balance between the adsorption characteristic and the oxidation characteristic of the detection electrode 22.

The adsorption characteristic and oxidation characteristic of the detection electrode 22 affect the sensitivity of the detection electrode 22 to ammonia. By making the sensitivity of the detection electrode 22 high and making variation with time of the sensor output of the detection electrode 22 unlikely, detection errors in the sensor output of the detection electrode 22 can be made less liable to occur, and the accuracy of detecting the ammonia concentration by the detection electrode 22 can be enhanced.

Hence, the ammonia sensor 1 of this embodiment can provide improved accuracy of detecting the ammonia concentration.

The adsorption characteristic of the detection electrode 22 to ammonia becomes lower as the content proportion of Pd with respect to Au on the surface of the detection electrode 22 increases. Decrease in the adsorption characteristic of the detection electrode 22 can be suppressed, and the sensitivity of the detection electrode 22 maintained at a high level, by making the content proportion of Pd with respect to Au on the surface of the detection electrode 22 as low as possible. The content proportion of Pd with respect to Au on the surface of the detection electrode 22 can be 0.5 mol % or more.

On the other hand, variation with time of the sensor output of the detection electrode 22 tends to occur in the positive direction when the adsorption characteristic of the detection electrode 22 with respect to ammonia is high, and tends to occur in the negative direction when the oxidation characteristic becomes high. If large variations with time of the sensor output of the detection electrode 22 arise in the positive direction or in the negative direction, then a difference is liable to occur between the sensor output immediately after the ammonia concentration has changed to some specific concentration and the sensor output after some time has elapsed since the ammonia concentration became changed.

The variation with time of the sensor output of the detection electrode 22 becomes smallest when the content proportion of Pd with respect to Au on the surface of the detection electrode 22 is of the order of 20 to 40 mol %. To minimize the variation with time, the content proportion of Pd with respect to Au on the surface of the detection electrode 22 can be made 40 mol % or less.

### Second Embodiment

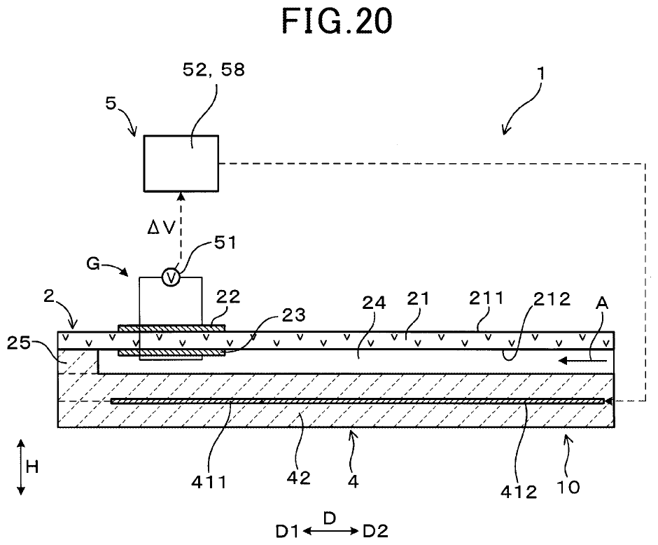

This embodiment illustrates a sensor element 10 that does not incorporate the oxygen element section 3. If the ammonia sensor 1 is required to detect only the ammonia concentration, then as shown in FIG. 20, the sensor element 10 can be formed by stacking together a first solid electrolyte body 21 that is provided with a detection electrode 22 and a reference electrode 23, an insulator 25 in which a reference gas duct 24 is formed, and an insulator 42 in which a heating body 41 is embedded. This embodiment has a single solid electrolyte body, designated as the first solid electrolyte body 21, provided with a detection electrode 22 and a reference electrode 23.

The detection electrode 22 is disposed on the first surface 211, which constitutes the outer surface of the first solid electrolyte body 21 and is exposed to the detection target gas G, while the reference electrode 23 is disposed in the reference gas duct 24. The configuration of the detection electrode 22 and the reference electrode 23, in this case, can be the same as for the first embodiment. Furthermore, in this case it would be possible for the ammonia sensor 1 to use the oxygen concentration measured by another gas sensor, in obtaining the ammonia concentration.

Other configuration features, operational effects, etc. of the ammonia sensor 1 of this embodiment are the same as those of the first embodiment. Furthermore, with this embodiment, components indicated by the same reference signs as those shown for the first embodiment are the same components as in the first embodiment.

### Embodiment 3

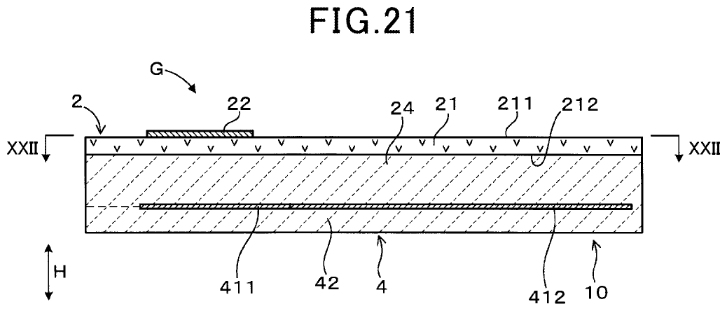

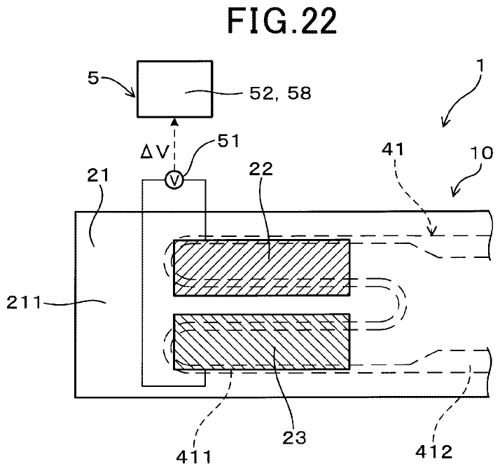

This embodiment illustrates a sensor element 10 that does not include the oxygen element section 3 and the reference gas duct 24. As shown in FIGS. 21 and 22, if there is not a reference gas duct 24 having a reference electrode 23 disposed therein, the detection electrode 22 and the reference electrode 23 can be disposed on the first surface 211 of the first solid electrolyte body 21, which constitutes the outermost surface of the sensor element 10. In that case, the concentration of ammonia in the detection target gas G can be detected based on the difference in catalytic activity for ammonia between the detection electrode 22 and the reference electrode 23. Also in that case, the configurations of the detection electrode 22 and the reference electrode 23 can be the same as for the first embodiment.

Other configuration features, operational effects, etc. of the ammonia sensor 1 of this embodiment are the same as those of the first and second embodiment. Furthermore, with this embodiment, components indicated by the same reference signs as those shown for the first embodiment are the same components as in the first and second embodiment.

The present disclosure is not limited to the embodiments, and it would be possible to configure different embodiments without departing from the gist of the disclosure. In addition, the present disclosure includes various modifications, including modifications that are within a scope of equivalents, and the like. Furthermore, the technical concepts of the present disclosure also include combinations, forms, etc. of various components that can be envisaged from the present disclosure.

What is claimed is:

1. An ammonia sensor comprising:
an ammonia element section, having an oxygen ion conductive solid electrolyte body, a detection electrode that is provided on a surface of the solid electrolyte body and which is exposed to a detection target gas, and a reference electrode provided on a surface of the solid electrolyte body,
a heater section having a heat generating section that generates heat when energized, and which heats the solid electrolyte body, the detection electrode, and the reference electrode by the heat generated from the heat generating section, and
a potential difference detection section that detects occurrence of a potential difference which arises between the detection electrode and the reference electrode, when the electrochemical reduction reaction of oxygen contained in the detection target gas and the electrochemical oxidation reaction of ammonia contained in the detection target gas in the detection electrode are balanced;
wherein:
the detection electrode contains at least Au and Pd, and
the content ratio of Au and Pd within 1 μm range of depth from the outermost face of the detection electrode in a direction perpendicular to a surface of the solid electrolyte body is 0.5 mol % or more and 40 mol % or less of Pd with respect to 100 mol % of Au.

2. The ammonia sensor according to claim 1, further comprising:
an oxygen element section having
an other solid electrolyte body, which has oxygen ion conductivity,
a gas chamber formed between the first-mentioned solid electrolyte body and the other solid electrolyte body, into which the gas to be detected is introduced through a diffusion resistance section,
a pump electrode and a NOx electrode provided on a surface of the other solid electrolyte body and housed in the gas chamber, exposed to the detection target gas, and
an other reference electrode, provided on a surface of the other solid electrolyte body that is opposite the surface on which the pump electrode and the NOx electrode are provided;
the ammonia sensor further comprising
a pumping section that pumps oxygen which is in the detection target gas in the gas chamber, by applying a DC voltage between the pump electrode and the other reference electrode,
a pump current detection section that detects a direct current flowing between the pump electrode and the other reference electrode, and
a NOx detection section that applies a DC voltage between the NOx electrode and the other reference electrode and detects a DC current flowing between the NOx electrode and the other reference electrode;
wherein
the ammonia sensor is configured to correct an ammonia concentration that is obtained based on the potential difference detected by the potential difference detection section using an oxygen concentration obtained based on the DC current detected by the pump current detection section in obtaining an ammonia output concentration, and to obtain a NOx concentration based on the DC current that is detected by the NOx detection section.
